(19) United States
(12) Patent Application Publication
(10) Pub. No.: US 2007/0243574 A1
(43) Pub. Date: Oct. 18, 2007

(54) Vascular mimic for drug and device evaluation

(75) Inventors: Stuart K. Williams, Tucson, AZ (US); Kristen O'Halloran Cardinal, Tucson, AZ (US)

Correspondence Address: THELEN REID BROWN RAYSMAN & STEINER LLP, P. O. BOX 640640, SAN JOSE, CA, 95164-0640, US

(73) Assignee: University of Arizona, Tucson, AZ (US)

(21) Appl. No.: 11/699,680

(22) Filed: Jan. 29, 2007

Related U.S. Application Data
(60) Provisional application No. 60/763,125, filed on Jan. 27, 2006.

(51) Int. Cl. C12Q 1/02; C12M 1/00; A01N 1/00; C12N 5/02

(52) U.S. Cl. 435/29; 435/284.1; 435/400; 623/916

(57) ABSTRACT

The present invention provides tissue engineered vascular grafts (TEVGs) and Blood Vessel Mimics (BVMs) and methods for using TEVGs as BVMs in in vitro model systems for the evaluation of intravascular devices and drugs. The present invention additionally relates to devices and methods for preparing TEVGs, BVMs and BVM model systems.

This application claims priority to U.S. Patent Application Ser. No. 60/763,125, filed Jan. 27, 2006, and U.S. patent application Ser. No. 11/314,281, filed Dec. 22, 2005, both of which are incorporated herein by reference.

### FIELD OF THE INVENTION

The present invention relates to tissue engineered vascular grafts (TEVGs) and Blood Vessel Mimics (BVMs) and methods for using TEVGs as BVMs in in vitro model systems for the evaluation of intravascular devices and drugs. The present invention additionally relates to devices and methods for preparing TEVGs, BVMs and BVM model systems.

### BACKGROUND OF THE INVENTION

Vascular and cardiac treatments continue to evolve and change as research advances and new technologies are developed. Stent technology, for example, is constantly changing as new modifications and coating technologies become available. Drug treatments are likewise constantly evolving. Stents can be drug-eluting, protein or polymer coated, or modified via numerous other methods. Surface treatments are crucial to the cellular response in the vessel, and to the overall success of stent function.

These constantly evolving drugs and devices possess great potential, but need to be evaluated before clinical applications are possible. Thus, an urgent need exists for an in vitro means to provide accurate and rapid initial assessment of intravascular treatment modalities (e.g., drugs and devices) prior to the initiation of in vivo/animal studies.

A number of publications describe methods and approaches for creating tissue engineered blood vessels for in vivo implantation. For example, L'Heureux, et al. A human tissue-engineered vascular media: a new model for pharmacological studies of contractile responses. FASEB J 15, 515, 2001, describe a method for creating a tissue engineered construct that can replace animal tissues currently used for pharmacologic studies. Prasad, et al. Survival of endothelial cells in vitro on Paclitaxel-loaded coronary stents. J Biomater Appl 19, 271, 2005, describe assessing endothelial response to stents in vitro, by exposing the stent to a homogenous cell solution. Sprague, et al. Human aortic endothelial cell migration onto stent surfaces under static and flow conditions. J Vasc Interv Radiol 8, 83, 1997, and Sprague, et al. Endothelial cell migration onto metal stent surfaces under static and flow conditions. J Long Term Eff Med Implants 10, 97, 2000, describe and utilize a 2-dimensional surface of endothelial cells under flow in a parallel plate to evaluate the cell migration onto square flat pieces of metallic material. Tremblay, et al. In vitro evaluation of the angiostatic potential of drugs using an endothelialized tissue-engineered connective tissue. J Pharmacol Exp Ther 315, 510, 2005, disclose using a tissue engineered construct composed of endothelial cells and a collagen sponge, to assess angiostatic potential by looking at capillary structures. Finally, Yeh, et al. Comparison of endothelial cells grown on different stent materials. J Biomed Mater Res A 76, 835, 2006, describe an in vitro approach to evaluate endothelial cells on a stent surface by directly sodding HUVECs directly onto flat metallic sheets. However, none of these publications, nor any other available research, describes the creation of a tissue engineered, three dimensional blood vessel mimic in vitro model system which can be used to evaluate intravascular devices such as stents as well as therapeutic agents.

The inventors have created an in vitro blood vessel mimic (BVM) model system that can be used to initially test and evaluate newly emerging intravascular devices and therapeutic agents.

### SUMMARY OF THE INVENTION

The present invention is based, in part, on the inventors' discovery that specifically designed tissue engineered vascular grafts (TEVGs) can be effectively used as model blood vessel mimics (BVMs) for the testing and evaluation of various treatment modalities including, but not necessarily limited to, therapeutic agents and an array of therapeutic intravascular devices. Accordingly, in one aspect, the present invention provides tissue engineered vascular grafts and/or BVMs for use in an in vitro model system comprising a tubular polymeric structure, wherein the luminal surface of the tubular structure comprises at least one layer of cells. The TEVGs of the present invention may comprise cells of any mammalian cell type. Additionally, the cells used in the TEVGs and BVMs of the present invention may include neoplastic and genetically modified cells. The polymeric graft scaffold structure used in the present invention may be comprised of either degradable and non-degradable polymers. In a particular embodiment, the polymeric graft scaffold structure comprises ePTFE.

In an additional aspect of the present invention, methods for preparing blood vessel mimics for use in an in vitro model system are also provided. By way of non-limiting example, such methods include providing a tubular polymeric structure; applying a low pressure transmural flow of a suspension of cells through the structure for a duration sufficient to adhere the cells to the luminal surface of the structure; and cultivating the blood vessel mimic in an in vitro environment for a duration sufficient to establish at least one cellular layer on the luminal surface of the structure. The pressure used may be from about 10 mmHg to about 55 mmHg and is preferably from about 35 mmHg to about 50 mmHg. In another embodiment, the BVM is cultivated in the in vitro environment from about 3 days to about 4 weeks.

The BVM may comprise cells of any mammalian cell type, and may comprise neoplastic and genetically engineered cells. In a preferred embodiment, the BVM comprises microvascular endothelial cells (MVECs) derived from adipose tissue. In another embodiment, the tubular polymeric structure is pretreated with a material selected from the group consisting of protein and plasma.

Also provided are methods for evaluating the cellular response to an intravascular device by, for example, deploying the intravascular device into a blood vessel mimic; cultivating the blood vessel mimic in an in vitro environment for a duration sufficient to allow for a cellular response to the intravascular device; and assessing the surface of the intravascular device and, optionally, the cells covering the luminal surface of the blood vessel mimic. The intravascular device used in the method of the invention may be any device, including stents (including drug eluting stents), stent grafts, catheters, pacemaker components, leads, sensors, filters, sutures, staples, patches, imaging systems, drug delivery devices, and combinations thereof. In another embodiment, the imaging system is selected from the group consisting of intravascular ultrasound, optical coherence tomography (OCT), laser induced fluorescence, and confocal imaging.

Additionally, methods for evaluating the cellular response to a therapeutic agent are provided. By way of example, such methods may comprise contacting the luminal surface of a blood vessel mimic with an effective amount of the therapeutic agent; cultivating the blood vessel mimic in an in vitro environment for a duration sufficient to allow for a cellular response to the agent; and evaluating the cells covering the luminal surface of the blood vessel mimic. In a particular embodiment, the blood vessel mimic may be analyzed in vitro using optical coherence tomography (OCT).

The present invention also provides devices for use in preparing blood vessel mimics comprising a media reservoir having an inlet and an outlet; a vessel chamber for holding a graft substrate having an inlet, an outlet and a cell-sodding port; a media flow loop connecting the vessel chamber and the media reservoir; and a pump configured to cause flow through the media flow loop. In one embodiment, the pump is a peristaltic pump. In an additional embodiment, the device comprises a port for introduction of a device.

### BRIEF DESCRIPTION OF THE DRAWINGS

The present invention will be understood more fully from the detailed description given below and from the accompanying drawings of various embodiments of the invention, which, however, should not be taken to limit the invention to the specific embodiments, but are for explanation and understanding only.

FIG. 1 provides a schematic of a bioreactor system configuration in accordance with one embodiment of the present invention, showing flow from the vessel chamber to the media reservoir and through a pump. An external cell-sodding port, and ePTFE vessel is also provided.

FIG. 2 is a perspective view of the bioreactor system according to one embodiment of the present invention.

FIG. 3 shows (A) a side view of a blood vessel mimic (BMV) according to one embodiment of the invention, including an polymeric (ePTFE) tubular scaffold structure with a cell layer on its luminal surface; and OCT images of the blood vessel mimic at (B) 4 weeks and (C) 5 weeks in the in vitro environment of the bioreactor.

FIG. 4 (A) shows stent deployment into the in vitro model system; and (B) shows a radiogram of a stent fully deployed in the BMV.

FIG. 5 shows cross-sectional and en face images of bisbenzimide (BBI) stained BMVs. Seven days post deployment, a cellular response to the bare metal stents was observed. (a) shows a cross-section of unstented BM; (b) shows a cross-section of a stented BVM; (c) shows an en face image of unstented BVM; and (d) shows an en face image of a stented BVM. Multiple images similar to (d) were acquired per sample and were used for quantifying cell coverage.

FIG. 6 provides a graph depicting the distribution of cell densities on bare metal stents. The number of cells per mm² of stent strut surface area was calculated from BBI en face images. The majority of stent segments had coverage between 50-250 cells/mm². No segments of the bare metal stent struts exhibited less than 50 cells/mm² after 7 days.

FIG. 7 provides a scanning electron micrograph of endothelial cell cobblestone morphology on the lumen of the BVM.

FIG. 8 shows histologic and immunohistochemical staining of 14 day BVMs. (a) H&E stain illustrates the basic structure of the cellular lining; (b) positive vWF staining was used to identify endothelial cells as the luminal monolayer; (c) alpha-smooth muscle actin antibodies identified smooth muscle cells interspersed directly beneath the endothelial cell lining; and (d) the majority of cells in the sub-endothelial layers stained positive for vimentin.

FIG. 9 depicts BVM response to bare metal stent (each OTC image is 10 mm×1.6 mm). (A) a thin cellular lining was present before stent deployment. Arrows delineate the thin hyperintense layer; (B) the post-deployment stent struts were visualized by a bright reflection followed by a dark vertical band. Disruption of cellular lining was visualized (arrows); (C)-(D) Areas of cellular accumulation upon the stent struts were seen in the 3- and 7 day images (arrows); and (E) extensive cellular accumulation was observed at day 14.

### DETAILED DESCRIPTION OF THE INVENTION

Embodiments of the present invention are described herein in the context of compositions, methods, systems and devices for providing blood vessel mimics for use in in vitro evaluation of therapeutic agents and devices. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

In one aspect, the present invention provides an in vitro model system comprising, an in vitro environment, and a blood vessel mimic, wherein the blood vessel mimic comprises at least one layer of cells.

The present invention also provides tissue engineered vascular grafts (TEVGs) and/or BVMs for use in an in vitro model system. In an embodiment, the TEVGs/BVMs comprise a tubular polymeric structure, wherein the luminal surface of the tubular structure comprises at least one layer of cells.

The cells to be adhered to the tubular polymeric structure (i.e. the graft substrate or scaffold structure) may include, for example, fibroblasts, smooth muscle cells, pericytes, macrophages, monocytes, plasma cells, mast cells, adipocytes, tissue-specific parenchymal cells, endothelial cells, urothelial cells, and various other cell types encountered in tissue engineering applications, including undifferentiated adult stem cells from various tissue sources. In a preferred embodiment, the cells are endothelial cells, and more preferably human microvascular endothelial cells obtained from microvascular rich adipose tissue as referred to in U.S. Pat. Nos. 4,820,626 (by Williams et al., issued Apr. 11, 1989), 5,230,693 (by Williams et al., issued Jul. 27, 1993), and 5,628,781 (by Williams et al., issued May 13, 1997), each of which is hereby incorporated by reference herein. The adherent cells may also include neoplastic (i.e., cancer) cells, as well as genetically modified cells.

In certain embodiments, tissue graft or cell suspensions further comprise at least one genetically engineered cell. In certain embodiments, tissue graft or cell suspensions comprising at least one genetically engineered cell will constitutively express or inducibly express at least one gene product encoded by at least one genetically engineered cell due to the genetic alterations within at least one genetically engineered cell induced by techniques known in the art. Descriptions of exemplary genetic engineering techniques can be found in, among other places, Ausubel et al., Current Protocols in Molecular Biology (including supplements through March 2002), John Wiley & Sons, New York, N.Y., 1989; Sambrook et al., Molecular Cloning: A Laboratory Manual, 2.sup.nd Ed., Cold Spring Harbor Laboratory Press, Cold Spring Harbor, N.Y., 1989; Sambrook and Russell, Molecular Cloning: A Laboratory Manual, 3.sup.rd Ed., Cold Spring Harbor Laboratory Press, Cold Spring Harbor, N.Y., 2001; Beaucage et al., Current Protocols in Nucleic Acid Chemistry, John Wiley & Sons, New York, N.Y., 2000 (including supplements through March 2002); Short Protocols in Molecular Biology, 4.sup.th Ed., Ausbel, Brent, and Moore, eds., John Wiley & Sons, New York, N.Y., 1999; Davis et al., Basic Methods in Molecular Biology, McGraw Hill Professional Publishing, 1995; Molecular Biology Protocols (see the highveld.com website), and Protocol Online (protocol-online.net). Exemplary gene products for genetically modifying the genetically engineered cells of the invention include plasminogen activator, soluble CD4, Factor VIII, Factor IX, von Willebrand Factor, urokinase, hirudin, interferons, including alpha-, beta- and gamma-interferon, tumor necrosis factor, interleukins, hematopoietic growth factor, antibodies, glucocerebrosidase, adenosine deaminase, phenylalanine hydroxylase, human growth hormone, insulin, erythropoietin, VEGF, angiopoietin, hepatocyte growth factor, PLGF, and the like.

The polymeric structure (scaffold) materials used in the present invention may be any preferably permeable material of various sizes and geometries. The material may be degradable or non-degradable. The material may be natural or synthetic materials, including, but not limited to, expanded poly-tetrafluoroethylene (ePTFE), polyurethane, polypropylene, polyethylene, polyamides, nylon, polyethylene terephthalate, polyethyleneterathalate, polycarbonate, polystyrene, polylactic acid, polyglycolic acid, a PLA/PGA mixture, dextran, polyethylene glycol, polycaprolactone, stainless steel, titanium/nickel alloys, silicone, and combinations thereof. In another embodiment, the graft scaffold may be a biopolymer, such as collagen. The material may be preclotted and/or elastin, or allograft vessels, such as cryopreserved vein, decellularized vein or artery. In yet another embodiment, the scaffold may be a composite material such as an elastin scaffold with a polymeric coating, for example electrospun on the surface to improve mechanical properties. The material may be pre-clotted or pre-treated with a protein (e.g., albumin) or plasma, which in certain embodiments can serve to further enhance the adherence, spreading, and growth of tissue cells on the substrate material. The graft substrate structure scaffolds may be constructed by any suitable method, including, but not limited to, those referred to in Liu, T. V. et al., 2004, Adv. Drug. Deliv. Rev. 56(11):1635-47; Nygren, P. A. et al., 2004, J. Immunol. Methods 290(1-2):3-28; Hutmacher, D. W. et al., 2004, Trends Biotechnol. 22(7):354-62; Webb, A. R. et al., 2004, Expert Opin. Biol. Ther. 4(6):801-12; and Yang, C. et al., 2004, BioDrugs 18(2):103-19.

The present invention also provides methods for preparing a blood vessel mimic for use in an in vitro model system. In a particular embodiment, the method consists of providing a tubular polymeric structure; applying a low pressure transmural flow of a suspension of cells through the structure for a duration sufficient to adhere the cells to the luminal surface of the structure (i.e., cell-sodding); and cultivating the blood vessel mimic in an in vitro environment for a duration sufficient to establish at least one cellular layer on the luminal surface of the structure. In one embodiment, the applied pressure is from about 10 mmHg to about 60 mmHg. In another embodiment, the pressure applied is from about 35 mmHg to about 50 mmHg. In still another embodiment, the pressure applied is about 50 mmHg.

In a further embodiment, a translumenal flow is applied through the tubular graft structure after the cells have adhered to the substrate. In a preferred embodiment, the rate of translumenal flow approximates a physiological flow rate.

The cell-sodding step of the method of the present invention may be carried out by injecting cells to be sodded into an aqueous perfusion media or by first creating a cellular suspension of the desired cells and employing this suspension as the perfusion media. In either case, a sustained low magnitude pressure will be applied to drive the suspended cells against a permeable substrate, for example, a tubular graft scaffold. The cellular slurry or suspension may be obtained by any suitable method known in the art, including culturing a quantity of cells and dispersing the cultured cells in media. In an embodiment, the suspension or slurry is prepared by harvesting adipose tissue via, for example, a liposuction technique, then mincing the tissue and subjecting the minced tissue to various enzymes, centrifugation, and resuspension to prepare an adipose-derived MVEC suspension. These and similar techniques can be applied to non-adipose tissue to prepare other types of cell suspensions or slurries for use in the sodding method of the present invention.

Prior to sodding the graft substrate may be pretreated with a protein, preferably albumin. The substrate also may be a preclotted or pretreated with a plasma. In certain embodiments such pretreatments can serve to further enhance the adherence, spreading, and growth of tissue cells on the substrate material.

In a particular embodiment of the present invention, the polymeric surface is pretreated with cells, and BVM is then cultivated in an in vitro environment for a duration sufficient to establish at least one layer of cells on the luminal surface of the structure. Then a second layer of cells is deposited on the luminal surface using a process of in situ cell sodding.

The tubular polymeric structure/graft substrate is then mounted in an apparatus capable of providing sustained low magnitude pressure to provide transmural flow of the cellular suspension in relation to the substrate. A preferred apparatus for vascular grafts are disclosed below, but any suitable apparatus may be used, provided it can hold the substrate in place while containing and subjecting the substrate to sustained transmural pressure gradients of between about 10 to 500 mmHg. In a preferred embodiment, the apparatus is a bioreactor further including mechanisms to provide translumenal flow, after deposition of the cells by transmural flow.

The term “sustained pressure” as used herein means pressure having a head of about 10 mmHg, about 15 mmHg, about 20 mmHg, about 25 mmHg and about 30 mmHg and about 55 mmHg, for about 5 min, about 20 min, about 30 min, about 40 min, about 50 min, about 1 hour, about 1.5 hours, about 2 hours, about 2.5 hours, about 3 hours, about 4 hours, about 5 hours or about 6 hours, to enhance the adhesion, growth and/or differentiation of the cells. One of ordinary skill in the art can select appropriate conditions for applying specific sustained pressures according to the types of cells, and substrate materials, and given the teachings herein.

The term “transmural pressure or flow” as used herein refers to pressure or flow from one side to the other side of a graft scaffold/polymeric structure, across the wall of the graft scaffold/polymeric structure. Where the graft scaffold/polymeric structure is a tubular graft scaffold, the transmural pressure flow is preferably from the lumen or intracapillary (“IC”) space of the graft to the outside or extracapillary (“EC”) space of the graft.

The term “translumenal pressure or flow” as used herein refers to pressure or flow longitudinally through the lumen of a tubular graft. The terms “translumenal flow” and “translumenal perfusion” may be used interchangeably. Translumenal perfusion may be applied, for example, after transmural flow, to provide a training or cleansing effect on the deposited cells. In this case, translumenal flow rates up to and including physiologic flow rates (up to about 160 ml/min) are preferred. Transmural flow rates as low as about ml/min according to the methods herein are sufficient to provide cellular adhesion capable of withstanding subsequent supraphysiologic flow.

The term “proximal” as used herein refers to a point of reference on the side of media inflow in relation to the center of a bioreactor.

The term “distal” as used herein refers to a point of reference on the side of media outflow in relation to the center of the bioreactor.

The term “intracapillary (IC)” refers to the lumen or the internal space of a tubular graft scaffold and may be interchangeably referred to as “intralumenal.”

The term “extracapillary (EC)” refers to the outside space of a tubular graft scaffold and may be interchangeably referred to as “extravascular” or “extralumenal.”

While the methods of the invention may be carried out in any suitable apparatus, the inventors have found that particular bioreactor designs and automated perfusion systems are especially well-suited to achieve optimal results in terms of consistent and uniform cell adherence and operator convenience.

The present invention additionally provides methods for evaluating the cellular response to a therapeutic agent and/or an intravascular device. In one embodiment, the method includes deploying the intravascular device into a blood vessel mimic of the present invention; cultivating the blood vessel mimic in a suitable in vitro environment for a duration sufficient to allow for a cellular response to the intravascular device; evaluating the surface of the intravascular device and, optionally, evaluating the cells covering the luminal surface of the blood vessel mimic.

The device evaluated by the methods of the present invention may include any intravascular device such as, for example, coated and uncoated stents, drug eluting stents, stent grafts, catheters, pacemaker components, leads, sensors, filters, sutures, staples, patches, imaging systems, drug delivery devices, and combinations thereof. In a particular embodiment of the present invention, the device is a stent.

In another embodiment, the imaging system evaluated by the methods of the present invention may include, by way of non-limiting example, intravascular ultrasound, optical coherence tomography, laser induced fluorescence, and confocal imaging systems.

In a particular embodiment, a method for evaluating the cellular response to a therapeutic agent is provided comprising contacting the luminal surface of a blood vessel mimic with an effective amount of the agent; cultivating the blood vessel mimic in an in vitro environment for a duration sufficient to allow for a cellular response to the agent; and evaluating the cells covering the luminal surface of the blood vessel mimic.

In accordance with particular embodiments of the invention, the therapeutic agent may include a any chemical composition, small molecule or drug, including a protein or nucleic acid. Formulations of the therapeutic agent of the present invention may be prepared by methods well-known in the pharmaceutical arts. For example, a particular drug may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The therapeutic composition would be useful for administering the therapeutic agent to the in vitro model system, as discussed herein.

The therapeutic agent may include any pharmaceutically acceptable carrier known in the art. In an embodiment, the therapeutic agent is a drug, such as for example, a cardiac or vascular drug. In a particular embodiment, the therapeutic agent is encapsulated or otherwise attached to a nanoparticle carrier as described in Missirlis D. et al. Doxorubicin encapsulation and diffusional release from stable, polymeric, hydrogel nanoparticies. Eur J Pharm. Sci 29:120-129, 2006; Westedt U. et al. Deposition of nanoparticles in the arterial vessel by porous balloon catheters: localization by confocal laser scanning microscopy and transmission electron microscopy. AAPS Pharm Sci 4: E41, 2002; and Westedt U et al. Effects of different application parameters on penetration characteristics and arterial vessel wall integrity after local nanoparticle delivery using a porous balloon catheter. Eur J Pharm Biopharm 58:161-168, 2004. As used herein, the term “effective amount” refers to an amount of an agent capable of eliciting a detectable (i.e., measurable) change to the cells and/or cellular environment of the BVM. This amount may be readily determined by the skilled artisan.

Devices for use in preparing a blood vessel mimics and BMV model systems are also provided by the present invention. In one embodiment, such a device comprises a media reservoir having an inlet and an outlet; a vessel chamber for holding a graft substrate having an inlet, an outlet and a cell-sodding port; a media flow loop connecting the vessel chamber and the media reservoir; and a pump configured to cause flow through the media flow loop. In another embodiment, the device additionally includes an incubator. In still another embodiment, the device includes a cell-sodding apparatus in fluid communication with the cell-sodding port, and the pump is a peristaltic pump.

The media used in the devices and BVM in vitro model systems of the present invention may include, for example M199, M199E, PBS, Saline, and Divalent Free DPBS.

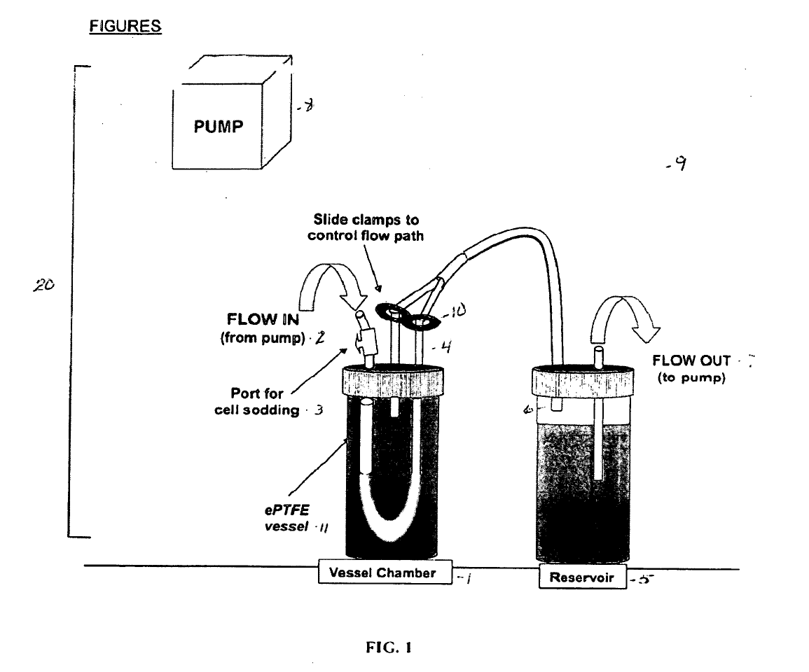

Reference is now made to FIG. 1 which illustrates one particular embodiment of a bioreactor system 20 that includes a vessel chamber 1 having an inlet 2 (containing a cell-sodding port 3) and an outlet 4, a media reservoir 5, having an inlet 6 and an outlet 7; a pump 8; and a media flow loop 9 connecting the vessel chamber, the media reservoir and the pump. In another embodiment, the bioreactor system optionally includes control clamps or valves 10 for use in controlling the media flow rate and path.

The pump 8 may be any suitable pump or combination of pumps, including, but not limited to, gear pumps, peristaltic pumps, diaphragm pumps, centrifugal pumps, and passive pressure heads created by a column of fluid. In a preferred embodiment the pump is a Watson-Marlow peristaltic pump. In one embodiment, the system is a vessel bioreactor, such as discussed below and in copending U.S. patent application Ser. No. 11/314,281 filed Dec. 22, 2005, which is herein incorporated by reference.

The system 20 allows for the deposition via pressure sodding of a desired fraction of mammalian cells onto a graft scaffold material. Williams S K, Rose D G, Jarrell B E. Microvascular Endothelial Cell Sodding of ePTFE Vascular Grafts: Improved Patency and Stability of Cellular Lining. J. Biomed. Materials Rsch. 1994; 28(2): 203-212. This may be accomplished within a laboratory setting in an automated fashion within a clinically feasible timeframe. A clinically feasible timeframe is generally considered to be from about 30 minutes to about 24 hours, depending upon a variety of factors, such as, for example, the types of cells, the amount of starting material, the amount of grafted cells needed, the time required for a maturation of the cell layer into a tissue, and the like. These factors are readily understood by a person having ordinary skill in the relevant art.

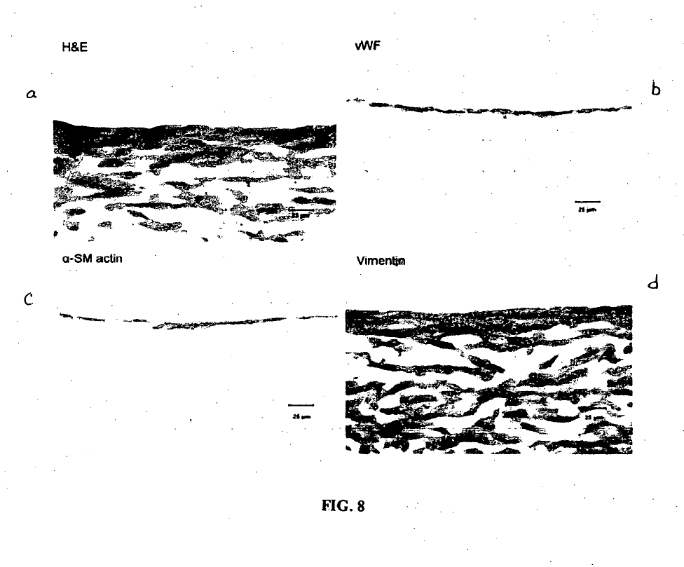

The system of the present invention additionally allows for the following to occur inside the system to maintain sterility: (1) cultivation of the BVM under flow to establish the cellular lining of the BVM; (2) deployment of an intravascular device or injection of a therapeutic agent into the BVM; and (3) imaging of the lumen of the BVM. In one embodiment, the bioreactor system of the present invention is place in a temperature controlled incubator (e.g., 37° C.), and media is circulated through the system at a physiologic flow rate for a period of time from about 3 days to 14 days to allow for the establishment of a cellular lining in the BVM. The establishment of a cellular lining can be verified using an array of methods well known to the skilled artisan. In one embodiment, cell lining development is verified using SEM and H&E staining. See e.g., FIGS. 7 and 8.

A sustained low-pressure gradient of at least 10 mmHg and not more than 500 mmHg may be used over a period from 30 seconds to 48 hours to deposit the cells upon the surface or in the graft scaffold material, depending upon the nominal pore size of the graft. The pressure gradient can be accomplished with any combination of positive and/or negative pressures such that the net gradient causes flow through the graft material.

The sodding media may be a commercially available media including DMEM, F12, AlphaMEM, University of Wisconsin Solution, etc., or any combination thereof, with or without additional factors, which may include heparin or other factors that accommodate the desired cell type.

As set forth herein, the bioreactor system holds the scaffold material 11 and can allow for flow of the media through a permeable scaffold.

In the case of a tubular scaffold, cells are deposited upon the luminal surface of the graft and the bioreactor holds the graft to allow for uniform cell deposition by virtue of uniform permeability along the long axis of the graft. Such a tubular graft may also by preloaded along its long axis to change the permeability of the graft, including opening up the pores of the graft material.

The illustrative system of FIG. 1 will typically include a microprocessor and associated software to control the system and automate one or more steps based on user input. The software may allow full or partial automation of, for example, controlling flow through tubular conduits by controlling pumps and valves, controlling temperature, and controlling cell separator and macerator devices. Preferably the system is fully automated, but capable of being reconfigured based on one or more input parameters. The systems may further include various sensors to detect or measure system parameters, such as pressures that would indicate a blockage, and signal same to the microprocessor or user.

While the automated methods of the invention may be carried out in any suitable apparatus, the inventors have found that particular bioreactor designs are especially well-suited to achieve optimal results in terms of consistent and uniform cell adherence and user convenience.

The permeable scaffold material may be mounted via the connectors to the IC proximal and distal tubing. In a specific embodiment, the bioreactor includes a stopcock attached to the proximal tubing via a divided connector, to allow for injection of cells into the bioreactor. In yet another specific embodiment, the bioreactor further includes at least one clamp or valve that can close either the distal EC tubing or the distal IC tubing to create, or shift between, transmural or translumenal pressure gradients, as explained below. In a preferred embodiment, each of the distal IC tubing and the distal EC tubing has its own valve or slide clamp.

Preferably, the vessels of the bioreactor are made of optically clear materials (e.g., polystyrene or polycarbonate) so that intra- and extra-luminal flow can be visually monitored. The bioreactor is preferably made from materials which are autoclavable, gamma, or gas-sterilizable. Furthermore, the bioreactor may contain a multiple silicone O-ring system, providing double seal contact for vessel attachment, so that the vessel length and angular position may be adjusted after a specimen is mounted between the two barbs within the bioreactor. In addition, metal thread inserts may be used to eliminate the need for threading manufactured components, and also eliminate the potential failure of plastic threads.

The configuration of the distal tubing which couples the distal IC and distal EC flow spaces, allows the user to switch between a transmural pressure gradient and a translumenal pressure gradient using the slide clamps, or in automated fashion within a bioreactor. This switch would typically take place to provide translumenal pressure after cell adhesion. In a specific embodiment, cells are introduced via stopcock or a septum connected via a divided connector to the proximal tubing. The distal IC slide clamp is then closed to allow only outflow from the EC space, thereby establishing a transmural pressure gradient from the proximal IC to distal EC space, and a small flux of media through the permeable scaffold while depositing adhering cells on the luminal surface and/or within the wall of the graft. The pressure gradient may be established either by generation of a positive pressure at the proximal IC side, a negative pressure at the distal EC side, or a combination of positive pressure at the proximal IC and negative pressure at the distal EC spaces. If desired, after cell adhesion to the luminal surface, the distal EC slide clamp may be closed and the distal IC slide clamp opened to allow flow through the lumen of the vessel to ensure cellular adhesion in the presence of a shear stress, which simulates a physiological environment.

The controlled, sustained differential pressure gradient across the permeable scaffold material may be created by any suitable configuration, including, but not limited to, gear pumps, peristaltic pumps, diaphragm pumps, centrifugal pumps, and passive pressure heads created by a column of fluid, so long as the pressure is sufficiently sustained and at a magnitude sufficient to achieve the advantages of the invention. In a particularly preferred embodiment, the pressure is applied transmurally to a vascular graft scaffold using media containing endothelial cells at a pressure head of about 50 mmHg and for a duration of about 5 minutes.

The methods and bioreactor apparatus of the present invention may be employed in combination with various media perfusion systems. The advantages of the invention may be optimized for certain tissue engineering applications by use of an automated cell culture apparatus, preferably as described in U.S. patent application Ser. Nos. 09/967,995 and 10/109,712.

The above-referenced patent applications provide automated perfusion culture platforms to provide controlled media flow, shear stress, nutrient delivery, waste removal, and improved mass transfer. These address many of the shortcomings of traditional culture systems by providing a sterile barrier to contamination while maintaining more uniform and controlled physiologic environments for cells and tissues, providing the user with sample access and data, and providing affordable reproducibility and reliability with data tracking and logging.

Such an automated perfusion system may include a durable cartridge containing a pump, valve array, flow meter, and user interface. It preferably has an embedded microcontroller and pre-programmed flow regimes with programmable flow states. Multiple perfusion loop cartridges may be housed within a single docking station rack, designed to be housed within a laboratory incubator. The disposable perfusion flowpath integrates with the cartridge and may have integrated media reservoirs, tubing for gas exchange, and a valve matrix controlling media flow. A bioreactor, in accordance with the present invention, may be mated with the flowpath. Periodic flow reversal can be employed to decrease differences in media composition from inlet to outlet. An automated sampling system also may be provided to allow the user to obtain a sample of media for analysis. Flow rates may vary from, for example, 1 up to 120 ml/min or more; flow may be monitored by an optical drop meter.

Because at least a portion of the flow for the current invention is typically transmural, the flow rate is dependent upon the permeability of the graft material, and decreases as the cells are applied to the luminal surface. Transmural flow rates before the introduction of cells can be from 5-50 ml/min depending on the graft material and generally decrease to 1-110 ml/min after the introduction of cells. Preferred endothelial cell numbers include 120,000-2,000,000 cells/cm² of luminal surface area, more preferably about 1,000,000 cells/cm².

The present invention is further described in the following examples, which are set forth to aid in the understanding of the invention, and should not be construed to limit in any way the scope of the invention as defined in the claims which follow thereafter.

### EXAMPLES

### Example 1

Materials and Methods

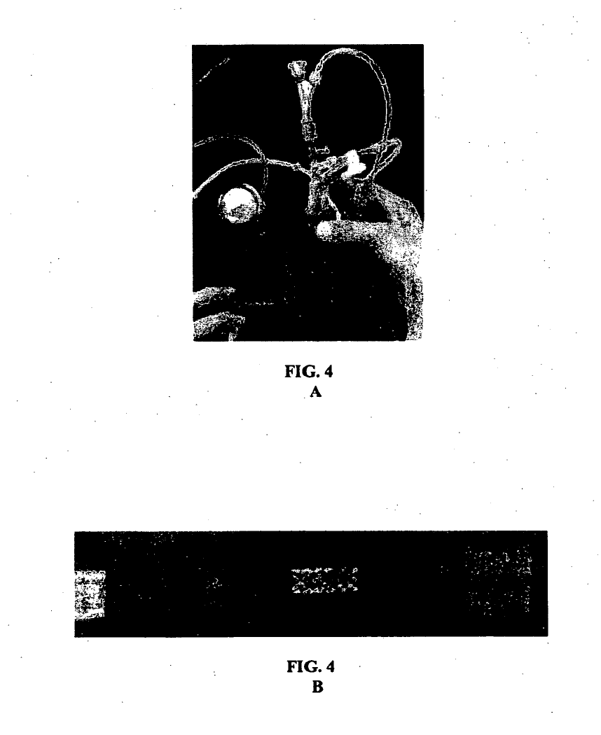

Stent surface modifications were evaluated in an in vitro blood vessel mimic model system. BVMs were created by pressure-sodding human microvessel endothelial cells onto the lumen of serum-conditioned 3 mm I.D. expanded polytetrafluoroethylene vascular grafts (C.R. Bard, Inc). Following cell sodding, BVMs were cultivated under flow in an in vitro environment in order to establish the cellular lining. After 1 week, stents were deployed into the BVM systems (see FIG. 4) via a catheter and introducer port.

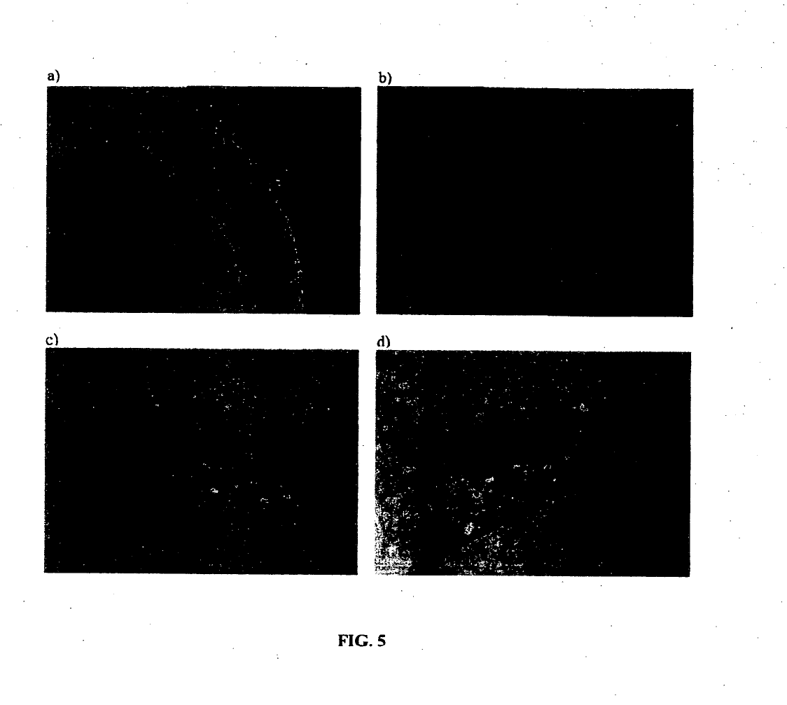

Following deployment of the stents, flow was continued for 1 week, at which point stented vessels were taken out of the system and fixed in 10% formalin. Vessels were cut longitudinally into three sections. Stent surface analysis was performed using scanning electron microscopy (SEM) to assess cell coverage and cell morphology. In addition, bisbenzimide (BBI) staining of cell nuclei provided information regarding endothelialization of the device surface (see FIG. 5). Hematoxylin and Eosin (H&E) staining of plastic embedded samples was used in order to assess the strut-associated cell interaction as well as the degree of neointimal thickening.

Results

After 1 week, the BVMs provided a model of stent-induced cellular response. SEM images were used for qualitative assessment of the stent surfaces and of cell morphology. Partial strut coverage was seen. BBI staining was performed to allow quantification of cell coverage. Cell nuclei fluoresced and were counted to calculate cell density on the stent struts. This type of evaluation provided useful information regarding endothelialization of the stent surface (see FIG. 5). Hematoxylin and Eosin staining, performed on plastic embedded sections, illustrated the neointimal response to stent implantation.

This example demonstrates that stent surface modifications can be evaluated in an in vitro BVM. Cell coverage of stent surfaces as well as neointimal development can be evaluated, and this analysis provides an assessment of the response of human cells to different surface modifications. The BVM system of the present invention permits rapid evaluation of stent designs and prototypes and provides a means for initial assessment of stent function prior to the initiation of in vivo animal studies.

### Example 2

Materials and Methods

Expanded polytetrafluoroethylene (ePTFE) of 3 mm and 4 mm inner diameter was cut into 4.5 cm lengths, steam sterilized, and denucleated. ePTFE grafts were conditioned with proteins by capping the grafts and forcing a serum dilution through the pores for 1 hour.

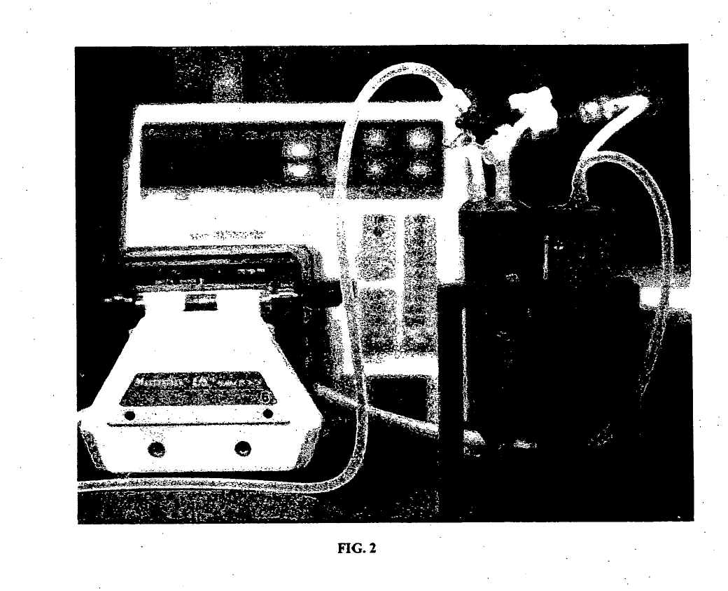

Conditioned grafts were placed in a bioreactor system, as shown in FIG. 2, and pressure-sodded with human microvessel endothelial cells (HMVECs), isolated from human liposuction fat. Transmural pressure was maintained for 1 hour to facilitate cell deposition.

Bioreactors were placed in a 37° C. incubator, and media was circulated luminally through each system at 15 mL/min for 10 days to allow for the establishment of a cellular lining. Developments of a cellular lining was verified with scanning electron microscopy (SEM) and hematoxytin and eosin (H&E) staining.

After 10 days of BVM development, flow was temporarily stopped to allow for stent deployment. Bare metal stents were introduced with sterile balloon catheter systems via an introducer port in each bioreactor system. Following proper catheter placement, the balloons were inflated to 5 atm to deploy the stents. Balloons were then deflated, the catheters were removed, and flow was resumed for 1 week to allow for a cellular response to the implanted devices.

After 1 week stented BVMs were evaluated with cell nuclear staining (3 mm vessels) or with optical coherence tomography (4 mm vessels). Vessels were fixed, cut in half longitudinally, and stained en face with bisbenzimlde (BBI). Samples were visualized under epifluorescence and images were acquired. Cell density was quantified by determining the average, number of cells/mm² on the stent surface.

The vessels were also imaged inside of the bioreactor chamber by optical coherence tomography (OCT). A 2 mm outer diameter end scope was inserted into the graft lumen for image acquisition, as shown in FIG. 4A, and OCT provided longitudinal images with a resolution of approximately 16 μm.

Results

SEM and H&E evaluation following BVM development showed the establishment of a luminal lining of cells. Specifically, SEM images illustrated a confluent, cobblestone morphology, and H&E staining verified the establishment of a luminal lining approximately 90-140 μm thick.

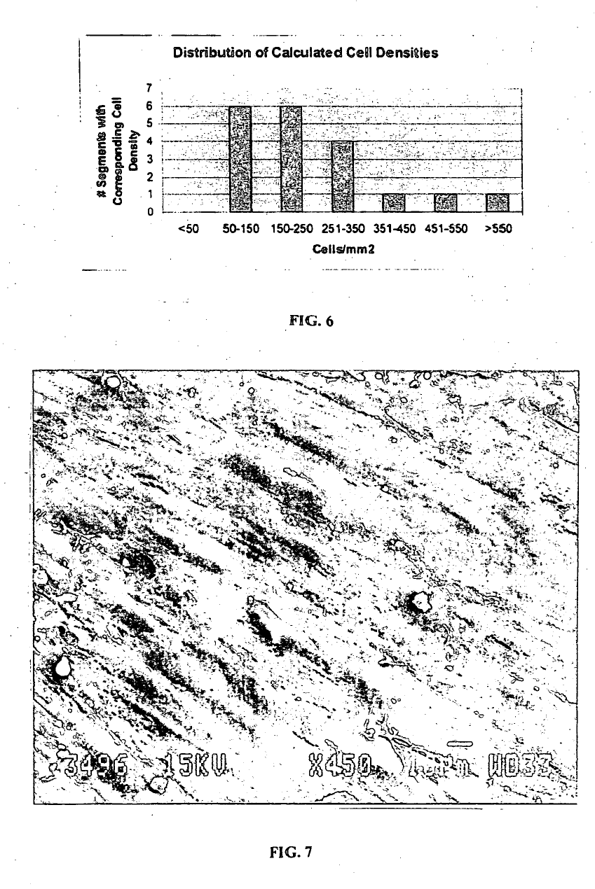

BBI stained samples were also viewed cross-sectionally (FIGS. 5A and B) and en face (FIGS. 5 C and D), in comparison with unstented vessels. Cross sections illustrated cellular growth over the stent struts. Further, En face images illustrated cellular migration onto the surface of the stents. After 1 week, the average cell coverage was 142 cells/mm² of stent surface area. See FIG. 6.

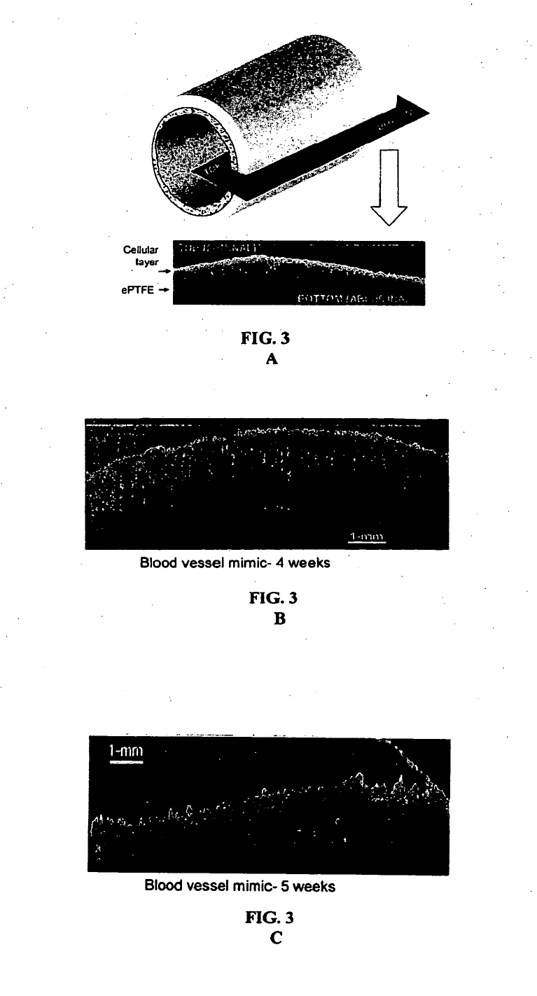

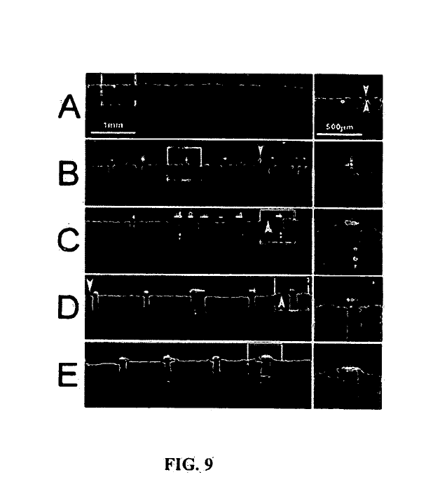

OCT images provided further evidence of the cellular response to the bare metal stent after 1 week of implantation. The BVM lumen between stent struts contained a cellular lining, distinct from the ePTFE scaffold. Cellular growth was seen in association with the stent struts, as shown in FIGS. 3 and 9.

These data demonstrate that the cellular response to stent implantation can be evaluated in an in vitro blood vessel mimic. Cell nuclear staining allows visualization and quantification of cell coverage over the stent surface. Additionally, OCT imaging allows minimally invasive, non-destructive evaluation of strut-associated cell growth. Human vascular cells utilized for the development of the BVM result in a human vessel mimic to assess stent surface modifications. The BVM provides an in vitro system to rapidly evaluate stent designs and prototypes for initial assessment of stent function prior to the initiation of animal studies.

### Example 3

Materials and Methods

The in vitro bioreactor system was developed with two chambers placed in series with tubing, and flow established with a Watson-Marlow peristaltic pump. The pump allowed flow regulation through the graft at rates ranging from 4-200 mL/min. The system permitted pressure-sodding of cells, stent deployment, and solute injection to take place internally to maintain sterility. 4 cm lengths of 3 mm I.D. expanded polytetrafluoroethylene (ePTFE, C.R. Bard, Inc.) vascular grafts were denucleated, treated with bovine serum, and inserted into the bioreactor.

Once each graft/bioreactor system was prepared, endothelial cells were freshly isolated through collagenase digestion of rat epididymal fat pads, and the cells were immediately pressure-sodded onto the grafts. Williams S K, Rose D G, Jarrell B E. Microvascular Endothelial Cell Sodding of ePTFE vascular grafts. Frontiers in Bioscience 2004; 9:1412-1421. Flow was increased to 6 mL/min and the constructs were maintained for 2-4 weeks at 37° C., with fresh media exchanged every 3 days. The development of a cellular lining on the construct lumen was evaluated using scanning electron microscopy (SEM). Additional imaging was performed using intravascular ultrasound and optical coherence tomography.

The feasibility of this model for the evaluation of stent deployment or drug delivery was analyzed in two ways. First, 3 mm coronary stents were deployed into the graft via a catheter and introducer port. (See FIG. 4). Each stent was deployed, followed by continuation of flow. Stent deployment capabilities and stent placement were also evaluated radiographically. Drug delivery was assessed through the injection of fluorescent solute dyes, which was analyzed with epifluorescent microscopy.

Results

Images taken with SEM established the ability to develop a confluent cell lining. Cell morphology on the ePTFE lumen was consistent with an endothelial cell monolayer. This model permitted stent deployment and drug delivery. Stents were successfully deployed onto the luminal surface of the graft. (See FIG. 4). Flow continued, with no signs of contamination, for up to 10 days post-deployment. Epifluorescent microscopy revealed a uniform delivery of fluorescent solute, which suggests that the TEVG model will permit successful drug delivery, thus allowing evaluation of drug effect on the intimal layer.

These data demonstrate that in addition to their role in the clinical setting, tissue engineered vascular grafts have potential as model blood vessel mimics for the testing of various treatment modalities. Confluent cell linings can be established in an in vitro environment that permits the introduction of both stents and drugs. This model will allow development and testing of newly emerging vascular therapies, including drug delivery and device deployment.

All publications, patents and patent applications mentioned in this specification are herein incorporated by reference into the specification to the same extent as if each individual publication, patent or patent application was specifically and individually indicated to be incorporated herein by reference.

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

What is claimed is:

1. An in vitro model system comprising an in vitro environment and a structure, wherein the structure comprises at least one layer of cells.

2. The system of claim 1, wherein the cells are selected from the group consisting of fibroblasts, smooth muscle cells, pericytes, macrophages, monocytes, plasma cells, mast cells, adipocytes, tissue-specific parenchymal cells, endothelial cells, urothelial cells, stem cells, and combinations thereof.

3. The system of claim 1, wherein the cells are endothelial cells.

4. The system of claim 3, wherein the cells are microvascular endothelial cells.

5. The system of claim 4, wherein the cells microvascular endothelial cells are derived from adipose tissue.

6. The system of claim 1, wherein the cells are neoplastic cells.

7. The system of claim 1, wherein at least one of the cells is a genetically modified cell.

8. An in vitro model system comprising an in vitro environment, and a blood vessel mimic, wherein the blood vessel mimic comprises at least one layer of cells.

9. The system of claim 8, wherein the cells are selected from the group consisting of fibroblasts, smooth muscle cells, pericytes, macrophages, monocytes, plasma cells, mast cells, adipocytes, tissue-specific parenchymal cells, endothelial cells, urothelial cells, stem cells, and combinations thereof.

10. The system of claim 8, wherein the cells are endothelial cells.

11. The system of claim 10, wherein the cells are microvascular endothelial cells.

12. The system of claim 11, wherein the cells microvascular endothelial cells are derived from adipose tissue.

13. The system of claim 8, wherein the cells are neoplastic cells.

14. The system of claim 8, wherein at least one of the cells is a genetically modified cell.

15. A tissue engineered vascular graft for use in an in vitro model system comprising: a tubular polymeric structure, wherein the luminal surface of the tubular structure comprises at least one layer of cells.

16. The tissue engineered vascular graft of claim 15, wherein the cells are selected from the group consisting of fibroblasts, smooth muscle cells, pericytes, macrophages, monocytes, plasma cells, mast cells, adipocytes, tissue-specific parenchymal cells, endothelial cells, urothelial cells, stem cells, and combinations thereof.

17. The tissue engineered vascular graft of claim 15, wherein the cells are endothelial cells.

18. The tissue engineered vascular graft of claim 15, wherein the cells are microvascular endothelial cells.

19. The tissue engineered vascular graft of claim 15, wherein the microvascular endothelial cells are derived from adipose tissue.

20. The tissue engineered vascular graft of claim 15, wherein the cells are neoplastic cells.

21. The tissue engineered vascular graft of claim 15, wherein at least one of the cells is a genetically modified cell.

22. The tissue engineered vascular graft of claim 15, wherein the polymeric structure comprises a material selected from the group consisting of elastin, ePTFE, collagen, polyurethane, polypropylene, polyethylene, polyamides, nylon, elastin, polyethylene terephthalate, polycarbonate, polystyrene, polylactic acid, polyglycolic acid, a PLA/PGA mixture, dextran, polyethylene glycol, polycaprolactone, stainless steel, titanium/nickel alloys, silicone, and combinations thereof.

23. The tissue engineered vascular graft of claim 15, wherein the polymeric structure comprises ePTFE.

24. A blood vessel mimic for use in an in vitro model system comprising: a tubular polymeric structure, wherein the luminal surface of the tubular structure comprises at least one layer of cells.

25. The blood vessel mimic of claim 24, wherein the cells are selected from the group consisting of fibroblasts, smooth muscle cells, pericytes, macrophages, monocytes, plasma cells, mast cells, adipocytes, tissue-specific parenchymal cells, endothelial cells, urothelial cells, stem cells, and combinations thereof.

26. The blood vessel mimic of claim 24, wherein the cells are endothelial cells.

27. The blood vessel mimic of claim 24, wherein the cells are microvascular endothelial cells.

28. The blood vessel mimic of claim 24, wherein the cells microvascular endothelial cells are derived from adipose tissue.

29. The blood vessel mimic of claim 24, wherein the cells are neoplastic cells.

30. The blood vessel mimic of claim 24, wherein at least one of the cells is a genetically modified cell.

31. The blood vessel mimic of claim 24, wherein the polymeric structure comprises a material selected from the group consisting of elastin, ePTFE, collagen, polyurethane, polypropylene, polyethylene, polyamides, nylon, elastin, polyethylene terephthalate, polycarbonate, polystyrene, polylactic acid, polyglycolic acid, a PLA/PGA mixture, dextran, polyethylene glycol, polycaprolactone, stainless steel, titanium/nickel alloys, silicone, and combinations thereof.

32. The blood vessel mimic of claim 24, wherein the polymeric structure comprises ePTFE.

33. A method of preparing a blood vessel mimic for use in an in vitro model system comprising:
providing a tubular polymeric structure;
applying pressure to a portion of the tubular structure, creating a transmural pressure gradient resulting in flow of fluid through the structure for a duration sufficient to permit deposition of cells, and adherence of cells to the luminal surface of the structure;
and cultivating the blood vessel mimic in an in vitro environment for a duration sufficient to establish at least one cellular layer on the luminal surface of the structure.

34. The method of claim 33, wherein the pressure is from about 10 mmHg to about 55 mmHg.

35. The method of claim 33, wherein the pressure is from about 35 mmHg to about 500 mmHg.

36. The method of claim 33, wherein the pressure is about 50 mmHg.

37. The method of claim 33, wherein the duration is of the pressure is about 30 seconds to about 60 minutes.

38. The method of claim 33, wherein the duration of cultivation is from about 3 days to about 4 weeks.

39. The method of claim 33, wherein the duration of cultivation is about 2 weeks.

40. The method of claim 33, wherein the cells are selected from the group consisting of fibroblasts, smooth muscle cells, pericytes, macrophages, monocytes, plasma cells, mast cells, adipocytes, tissue-specific parenchymal cells, endothelial cells, urothelial cells, stem cells, and combinations thereof.

41. The method of claim 33, wherein the cells are endothelial cells.

42. The method of claim 33, wherein the cells are microvascular endothelial cells.

43. The method of claim 33, wherein the cells microvascular endothelial cells are derived from adipose tissue.

44. The method of claim 33, wherein the cells are neoplastic cells.

45. The method of claim 33, wherein at least one of the cells is a genetically modified cell.

46. The method of claim 33, wherein the polymeric structure comprises a material selected from the group consisting of elastin, ePTFE, collagen, polyurethane, polypropylene, polyethylene, polyamides, nylon, elastin, polyethylene terephthalate, polycarbonate, polystyrene, polylactic acid, polyglycolic acid, a PLA/PGA mixture, dextran, polyethylene glycol, polycaprolactone, stainless steel, titanium/nickel alloys, silicone, and combinations thereof.

47. The method of claim 33, wherein the polymeric structure comprises ePTFE.

48. The method of claim 33, further comprising applying a translumenal flow through the tubular graft after the cells have adhered to the substrate.

49. The method of claim 33, wherein the translumenal flow is at a physiological flow rate.

50. The method of claim 33, wherein the polymeric structure is pretreated with a material selected from the group consisting of protein and plasma.

51. The method of claim 33, wherein the polymeric surface is pre-treated with cells, cultivating the blood vessel mimic in an in vitro environment for a duration sufficient to establish at least on cellular layer on the luminal surface of the structure and subsequently depositing a second layer of cells onto the luminal surface.

52. The blood vessel mimic of claim 51, wherein the cells are selected from the group consisting of fibroblasts, smooth muscle cells, pericytes, macrophages, monocytes, plasma cells, mast cells, adipocytes, tissue specific parenchymal cells, endothelial cells, urothelial cells, stem cells, and combinations thereof.

53. The blood vessel mimic of claim 51, wherein the cells are endothelial cells.

54. The blood vessel mimic of claim 51, wherein the cells are microvascular endothelial cells.

55. The blood vessel mimic of claim 51, wherein the cells are microvascular endothelial cells derived from adipose tissue.

56. The blood vessel mimic of claim 51, wherein the cells are neoplastic cells.

57. The blood vessel mimic of claim 51, wherein at least one of the cells is a genetically modified cell.

58. A method for evaluating the cellular response to an intravascular device comprising: deploying the intravascular device into a blood vessel mimic; cultivating the blood vessel mimic in an in vitro environment for a duration sufficient to allow for a cellular response to the intravascular device; and evaluating the surface of the intravascular device and, optionally, the cells covering the luminal surface of the blood vessel mimic.

59. The method of claim 58, wherein the intravascular device is selected from the group consisting of, stents, stent grafts, catheters, pacemaker components, leads, sensors, filters, sutures, staples, patches, imaging systems, drug delivery devices, and combinations thereof.

60. The method of claim 58 wherein the device is a stent.

61. The method of claim 60, wherein the stent is a drug eluting stent.

62. The method of claim 58, wherein the imaging system is selected from the group consisting of intravascular ultrasound, optical coherence tomography, laser induced fluorescence, and confocal imaging.

63. The method of claim 58, wherein the imaging system is optical coherence tomography.

64. The method of claim 58, wherein the duration of cultivation is from about 1 day to about 4 weeks.

65. The method of claim 58, wherein the duration of the cultivation is about 1 week.

66. A method for evaluating the cellular response to a therapeutic agent comprising: contacting the luminal surface of a blood vessel mimic with an effective amount of the agent; cultivating the blood vessel mimic in an in vitro environment for a duration sufficient to allow for a cellular response to the agent; and evaluating the cells covering the luminal surface of the blood vessel mimic.

67. The method of claim 66, wherein the therapeutic agent is a drug.

68. The method of claim 66 wherein the therapeutic agent is encapsulated in a nanoparticle carrier.

69. The method of claim 66 wherein the therapeutic agent is eluted from a stent.

70. The method of claim 66, wherein the evaluation comprises imaging the blood vessel mimic in vitro by optical coherence tomography.

71. The method of claim 66, wherein the duration of cultivation is from about 1 day to about 4 weeks.

72. The method of claim 66, wherein the duration of the cultivation is about 1 week.

73. An apparatus for use in preparing a blood vessel mimic comprising:
a media reservoir having an inlet and an outlet;
a vessel chamber for holding a graft substrate having an inlet, an outlet and a cell-sodding port;
a media flow loop connecting the vessel chamber and the media reservoir;
and a pump configured to cause flow through the media flow loop.

74. A blood vessel mimic model system comprising the apparatus of claim 73, a blood vessel mimic, and optionally, an incubator.

75. The apparatus of claim 743, further comprising a cell-sodding apparatus in fluid communication with the cell-sodding port.

76. The apparatus of claim 73, wherein the pump is a peristaltic pump.

77. The apparatus of claim 73, further comprising a port for introduction of a device.

78. The apparatus of claim 73, further comprising a heater.

79. The apparatus of claim 73, wherein the media is selected from the group consisting of M199, M199E, PBS, Saline, and Divalent Free DPBS.

80. The apparatus of claim 73, further comprising a device to control fluid flow between the vessel chamber and the media reservoir.

81. The apparatus of claim 73, wherein the device is a clamp.

82. The apparatus of claim 81, wherein the device is at least one valve.
